(19) United States
(12) Patent Application Publication
(10) Pub. No.: US 2021/0294943 A1
(43) Pub. Date: Sep. 23, 2021

(54) TPMS MICROSTRUCTURAL MATERIAL WITH HOLES AND OPTIMIZATION DESIGN METHOD THEREFOR

(71) Applicant: DALIAN UNIVERSITY OF TECHNOLOGY, Dalian, Liaoning (CN)

(72) Inventors: Zhan KANG, Dalian, Liaoning (CN); Jingcheng GAO, Dalian, Liaoning (CN); Yaguang WANG, Dalian, Liaoning (CN)

(21) Appl. No.: 17/257,772

(22) PCT Filed: Dec. 4, 2019
(86) PCT No.: PCT/CN2019/122952

§ 371 Date: Jan. 4, 2021

(30) Foreign Application Priority Data
Mar. 21, 2019 (CN) 201910215285.3

(51) Int. Cl. G06F 30/23; G06F 30/10; B33Y 80/00

(52) U.S. Cl. CPC G06F 30/23; G06F 2113/10; B33Y 80/00; G06F 30/10

(57) ABSTRACT

A TPMS microstructural material with holes and an optimization design method therefor are related to the technical fields of topology optimization, microstructural materials and 3D printing. TPMS is smooth, has a large specific surface area and good mechanical properties, and has a good application prospect in the field of microstructural material design. According to the optimization design method, an advanced design method, i.e., topology optimization, is applied to the microstructural material design based on TPMS. The method is to conduct topology optimization on a TPMS model, and then design the number, layout and shape of holes in a complete TPMS according to the results of topology optimization so as to obtain the microstructural material with different configurations. The material obtained has a higher utilization rate and a better lightweight property.

### TECHNICAL FIELD

The present invention belongs to the technical fields of topology optimization, microstructural materials and 3D printing, and relates to a TPMS microstructural material with holes and an optimization design method therefor.

### BACKGROUND

A topology optimization method is a new-type structural design method, which can be used to obtain an optimal material distribution in given constraints and design a novel structure from scratch. The method has been successfully applied to the design of microstructural materials such as materials with the characteristics of negative Poisson's ratio, zero thermal expansion, heat dissipation and energy absorption. TPMS is short for Triply Periodic Minimal Surface, and can be arrayed periodically along the three coordinate axes of a three-dimensional orthogonal rectangular coordinate system. The TPMS is smooth, has a large specific surface area and good mechanical properties, and has been applied to the fields of tissue engineering, energy absorption, heat dissipation, etc. by some researchers at present. However, the current design and research on the TPMS are all shells adopting a complete solid structure or based on a complete TPMS, and the application potential of materials has not been fully explored.

The present invention uses topology optimization technology to obtain an innovative configuration based on the TPMS, designs holes in a complete TPMS model, and obtains a microstructural material with a higher material utilization rate and a better structural lightweight property. The microstructural material designed can be manufactured by 3D printing technology.

### SUMMARY

In view of the defects of the existing microstructural materials based on TPMS, the present invention provides a TPMS microstructural material with holes and an optimization design method. First, topology optimization of the maximum tensile modulus and the maximum bulk modulus is conducted on a TPMS model, and then microstructural materials with different configurations are designed according to the results of topology optimization. The microstructural material with a relatively complex configuration designed by the present invention can be manufactured by 3D printing technology.

To achieve the above purpose, the present invention adopts the following technical solution:

A TPMS microstructural material with holes, which is designed with holes in a complete TPMS by the topology optimization method, and the thickness of the TPMS as well as the number, layout and shape of the holes can be adjusted. The holes are circular, triangular, quadrangular, pentagonal or hexagonal in shape.

An optimization design method for the TPMS microstructural material with holes, wherein the method is to conduct topology optimization of the maximum tensile modulus and the maximum bulk modulus on a TPMS model, then design the number, layout and shape of the holes in the complete TPMS according to the results of topology optimization so as to obtain the microstructural material with different configurations, and adjust the properties such as stiffness, strength and permeability of the microstructural material by further adjusting the size of the holes and the thickness of the TPMS. The method comprises the following specific steps:

Step 1: building a complete TPMS model according to a TPMS mathematical expression;

Building a complete P-type TPMS model according to a P-type TPMS, and an expression of the P-type TPMS is:

$$cos ( \frac{2 π x}{D} ) + cos ( \frac{2 π y}{D} ) + cos ( \frac{2 π z}{D} ) = 0 \\ x , y , z ∈ [ - D / 2 , D / 2 ]$$

Wherein x, y and z are coordinates of a three-dimensional orthogonal rectangular coordinate system; and D is a unit cell size of the microstructural material.

Step 2: considering the symmetry of the P-type TPMS model, taking a ⅛ TPMS as a design domain, applying symmetric boundary conditions to the symmetric planes of the model, and conducting topology optimization of the maximum tensile modulus and the maximum bulk modulus. The optimization formula is:

$$\{ \begin{matrix}find X = {( {x}_{1} , {x}_{2} , ⋯ , {x}_{n} )}^{T} \\ max f ( X ) \\ s . t . V ( X ) \leq {V}_{max}\end{matrix}$$

Wherein X is an element relative density, n is the number of design variables, an objective function f(X) is the tensile modulus or bulk modulus of the microstructural material, constraints are volume constraints, V(X) is the volume of the microstructural material, and Vmax is the maximum volume of the microstructural material designated.

Step 3: in order to obtain the microstructural material symmetric along the three axes of the three-dimensional orthogonal rectangular coordinate system, applying a rotationally symmetric constraint to a ⅛ P-type TPMS model.

Step 4: conducting topology optimization of the maximum tensile modulus and the maximum bulk modulus on the ⅛ TPMS model.

Step 5: designing holes in the TPMS according to the results of topology optimization, reconstructing the results of topology optimization to obtain microstructural material unit cells with different configurations, and further adjusting the size of the holes and the thickness of the TPMS to adjust the properties of the microstructural material. New-type tissue engineering scaffolds, energy absorption structures, etc. based on P-type TPMS are designed, and structures which can meet different needs are obtained by adjusting the size parameter of the microstructural material unit cells with various configurations, and combining and transforming the unit cells with various configurations according to actual engineering needs.

The configurations of the microstructural material unit cells include configuration I, configuration II, configuration III, configuration IV and configuration V. Each configuration is symmetric along the three axes of the three-dimensional orthogonal rectangular coordinate system and can be obtained by symmetry operation on the ⅛ model. The ⅛ model of each configuration is a rotationally symmetric model with three periods. Each configuration is designed with a closed ring structure 2-1 respectively at the tops in 6 directions, i.e., upwards, downwards, frontwards, backwards, leftwards and rightwards, to ensure a smooth transition between unit cells with different configurations. The details are as follows:

The ⅛ model of configuration I is designed with 3 identical closed triangular holes 2-2 and 3 identical open triangular holes 2-3, and the holes 2-3 become closed quadrangular holes 2-4 after symmetry operation in a complete configuration I.

The ⅛ model of configuration II is designed with 6 identical closed triangular holes 3-1, 3 identical closed quadrangular holes 3-2 and 3 identical open quadrangular holes 3-3, and the holes 3-3 become closed hexagonal holes 3-4 after symmetry operation in a complete configuration II.

The ⅛ model of configuration III is designed with 1 circular hole 4-1, 3 identical closed triangular holes 4-2, 6 identical closed fan-shaped holes 4-3 and 3 identical open triangular holes 4-4, and the holes 4-4 become closed quadrangular holes 4-5 after symmetry operation in a complete configuration III.

The ⅛ model of configuration IV is designed with 1 closed hexagonal hole 5-1, 6 identical closed quadrangular holes 5-2 and 6 identical open triangular holes 5-3, and the holes 5-3 become closed triangular holes 5-4 after symmetry operation in a complete configuration IV.

The ⅛ model of configuration V is designed with 3 identical closed pentagonal holes 6-1, 3 identical closed hexagonal holes 6-2 and 6 identical open quadrangular holes 6-3, and the holes 6-3 become closed pentagonal holes 6-4 after symmetry operation in a complete configuration V.

Further, the designed new-type structure is relatively complex and can be manufactured by 3D printing technology.

The present invention has the following beneficial effects: the topology optimization method is introduced into the design of the TPMS model, and a new-type microstructural material is obtained by designing holes in the complete TPMS. The TPMS microstructural material with holes have more adjustable parameters and higher design freedom, so that the application scope and practical effect of the TPMS model in structural design are improved. For example, when the new-type microstructural material designed is applied to the design of a tissue engineering scaffold, the permeability of the tissue engineering scaffold can be enhanced, the transport efficiency of nutrients and metabolic wastes in tissue engineering can be improved, the strength of the tissue engineering scaffold with a high porosity can be enhanced, and the mechanical properties of the tissue engineering scaffold can be controlled more accurately. Alternatively, the material can be used in thermal protection structures, active cooling structures, etc. in the field of aerospace. Compared with a complete TPMS microstructure, a TPMS microstructure with holes can achieve a lower microstructure equivalent density and achieve structural lightweight under the existing manufacturing capacity.

### DESCRIPTION OF DRAWINGS

FIG. 1 is a P-type TPMS model, wherein (a) is a front view thereof, (b) is an axial side view thereof, and (c) is a schematic diagram of a ⅛ model thereof.

FIG. 2 is a microstructural material configuration I designed based on a P-type TPMS, wherein (a) is a front view thereof, (b) is an axial side view thereof, (c) is a schematic diagram of a ⅛ model thereof, 2-1 is a closed ring connecting structure between microstructural material unit cells, and 2-2, 2-3 and 2-4 are holes designed.

FIG. 3 is a microstructural material configuration II designed based on a P-type TPMS, wherein (a) is a front view thereof, (b) is an axial side view thereof, (c) is a schematic diagram of a ⅛ model thereof, and 3-1, 3-2, 3-3 and 3-4 are holes designed.

FIG. 4 is a microstructural material configuration III designed based on a P-type TPMS, wherein (a) is a front view thereof, (b) is an axial side view thereof, (c) is a schematic diagram of a ⅛ model thereof, and 4-1, 4-2, 4-3, 4-4 and 4-5 are holes designed.

FIG. 5 is a microstructural material configuration IV designed based on a P-type TPMS, wherein (a) is a front view thereof, (b) is an axial side view thereof, (c) is a schematic diagram of a ⅛ model thereof, and 5-1, 5-2, 5-3 and 5-4 are holes designed.

FIG. 6 is a microstructural material configuration V designed based on a P-type TPMS, wherein (a) is a front view thereof, (b) is an axial side view thereof, (c) is a schematic diagram of a ⅛ model thereof, and 6-1, 6-2, 6-3 and 6-4 are holes designed.

FIG. 7 is a schematic diagram of a front surface of an array composed of a variety of TPMS microstructural materials with holes in the present invention.

FIG. 8 is a schematic diagram of a side surface of an array composed of a variety of TPMS microstructural materials with holes in the present invention.

### DETAILED DESCRIPTION

To be fully explain, the present invention will be further described in detail below in combination with the drawings and the embodiments. It should be understood that specific embodiments described herein are only used for explaining the present invention, not used for limiting the present invention.

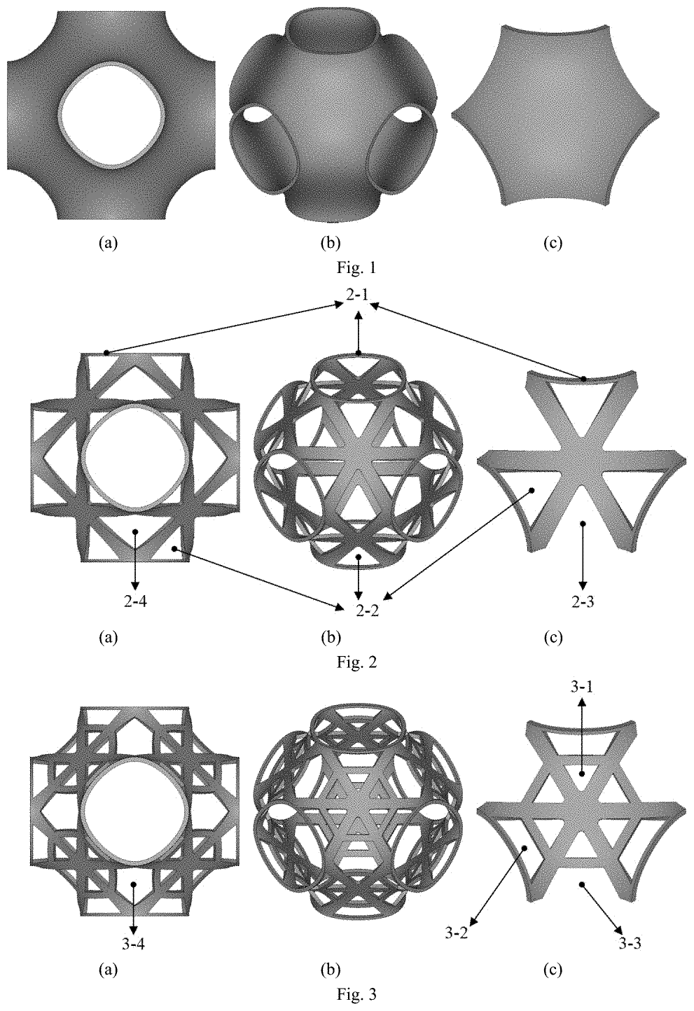

FIG. 1 shows a complete P-type TPMS model. Since the model has three symmetric planes, a ⅛ model as shown in FIG. 1(c) is taken to conduct topology optimization. In order to obtain the microstructural material symmetric along the three axes of the three-dimensional orthogonal rectangular coordinate system, a rotationally symmetric constraint is applied to the design domain shown in FIG. 1(c). In the topology optimization method of the present invention, the design variable is element relative density. Considering the volume constraints, the tensile modulus or bulk modulus of the microstructural material is maximized.

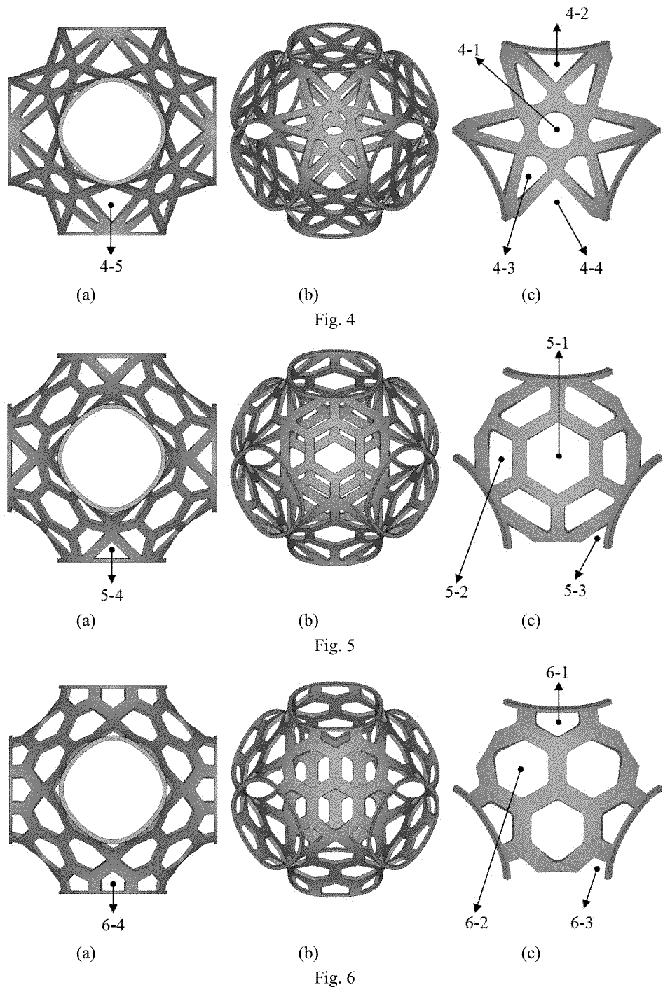

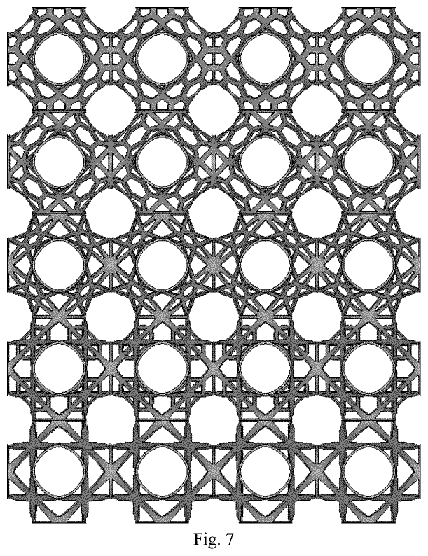

The holes as shown by 2-2 are designed on the complete P-type TPMS model according to the results of topology optimization, so as to obtain the microstructural material unit cells with different configurations as shown in FIG. 2 to FIG. 6. Unit cells of each configuration are designed with a closed ring structure as shown by 2-1 respectively at the interfaces of the tops in 6 directions, i.e., upwards, downwards, frontwards, backwards, leftwards and rightwards, to ensure a smooth transition between unit cells with different configurations.

Microstructural material unit cells with the five configurations shown in FIG. 2 to FIG. 6 can be manufactured by 3D printing technology.

Embodiment:

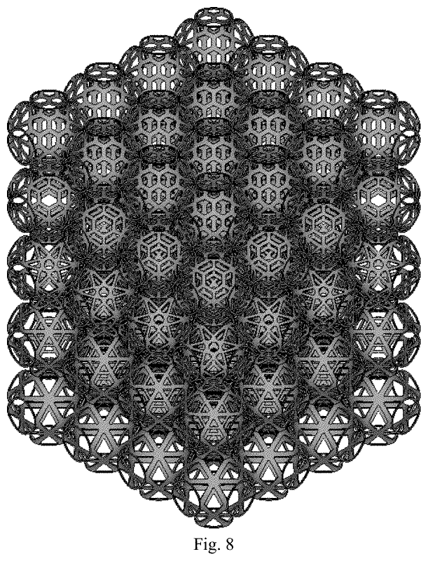

The TPMS microstructural material with holes provided by the present invention is widely used. For example, the material can be used for designing tissue engineering scaffolds. A new-type tissue engineering scaffold designed by the TPMS microstructural materials with holes as shown in FIG. 7 and FIG. 8 is obtained by combining microstructural materials with 5 different configurations, and is divided into 5 layers. The number of unit cells in each layer is 4×4, and the size of each unit cell is 1 mm×1 mm×1 mm. The holes of the microstructural materials in the scaffold can improve the lightweight performance of the scaffold, enhance the strength of the scaffold and enhance the permeability, etc. of the scaffold. A model file is obtained by a three-dimensional modeling software, an STL file which can be recognized by a 3D printer is exported, and printing is carried out based on a biodegradable material.

The above embodiments only express the implementation of the present invention, and shall not be interpreted as a limitation to the scope of the patent for the present invention. It should be noted that, for those skilled in the art, several variations and improvements can also be made without departing from the concept of the present invention, all of which belong to the protection scope of the present invention.

1. A TPMS microstructural material with holes, wherein the TPMS microstructural material is designed with holes in a complete TPMS by a topology optimization method, and the thickness of the TPMS as well as the number, layout and shape of the holes can be adjusted.

2. The TPMS microstructural material with holes according to claim 1, wherein the holes are circular, triangular, quadrangular, pentagonal or hexagonal in shape.

3. An optimization design method for the TPMS microstructural material with holes according to claim 1, wherein the method is to conduct topology optimization on a TPMS model, design the number, layout and shape of the holes in the complete TPMS according to the results of topology optimization so as to obtain the microstructural material with different configurations, and adjust the properties of the microstructural material by further adjusting the size of the holes and the thickness of the TPMS, which comprises the following steps:
step 1: building a complete TPMS model according to a TPMS mathematical expression as shown below:
$$cos ( \frac{2 π x}{D} ) + cos ( \frac{2 π y}{D} ) + cos ( \frac{2 π z}{D} ) = 0 \\ x , y , z ∈ [ - D / 2 , D / 2 ]$$
wherein x, y and z are coordinates of a three-dimensional orthogonal rectangular coordinate system; and D is a unit cell size of the microstructural material;
step 2: considering the symmetry of the TPMS model, taking a ⅛ TPMS as a design domain, applying symmetric boundary conditions to the symmetric planes of the model, and conducting topology optimization of the maximum tensile modulus and the maximum bulk modulus; and the optimization formula thereof is:
$$\{ \begin{matrix}find X = {( {x}_{1} , {x}_{2} , ⋯ , {x}_{n} )}^{T} \\ max f ( X ) \\ s . t . V ( X ) \leq {V}_{max}\end{matrix}$$
wherein X is an element relative density, n is the number of design variables, an objective function f(X) is the tensile modulus or bulk modulus of the microstructural material, constraints are volume constraints, V(X) is the volume of the microstructural material, and Vmax is the maximum volume of the microstructural material designated;
step 3: in order to obtain the microstructural material symmetric along the three axes of the three-dimensional orthogonal rectangular coordinate system, applying a rotationally symmetric constraint to a ⅛ P-type TPMS model;
step 4: conducting topology optimization of the maximum tensile modulus and the maximum bulk modulus on the ⅛ TPMS model; and
step 5: designing holes in the TPMS according to the results of topology optimization, reconstructing the results of topology optimization to obtain microstructural material unit cells with different configurations, and further adjusting the size of the holes and the thickness of the TPMS; the microstructural material unit cells have different configurations, and each configuration is symmetric along the three axes of the three-dimensional orthogonal rectangular coordinate system and can be obtained by symmetry operation on the ⅛ model; the ⅛ model of each configuration is a rotationally symmetric model with three periods; each configuration is designed with a closed ring structure respectively at the tops in 6 directions, i.e., upwards, downwards, frontwards, backwards, leftwards and rightwards, to ensure a smooth transition between unit cells with different configurations; and structures which can meet different needs can be obtained by combining and designing the microstructural material unit cells with one or more configurations.

4. The optimization design method according to claim 3 for the TPMS microstructural material with holes, wherein the configurations of the microstructural material unit cells include configuration I, configuration II, configuration III, configuration IV and configuration V:
the ⅛ model of configuration I is designed with 3 identical closed triangular holes 2-2 and 3 identical open triangular holes 2-3, and the holes 2-3 become closed quadrangular holes 2-4 after symmetry operation in a complete configuration I;
the ⅛ model of configuration II is designed with 6 identical closed triangular holes 3-1, 3 identical closed quadrangular holes 3-2 and 3 identical open quadrangular holes 3-3, and the holes 3-3 become closed hexagonal holes 3-4 after symmetry operation in a complete configuration II;
the ⅛ model of configuration III is designed with 1 circular hole 4-1, 3 identical closed triangular holes 4-2, 6 identical closed fan-shaped holes 4-3 and 3 identical open triangular holes 4-4, and the holes 4-4 become closed quadrangular holes 4-5 after symmetry operation in a complete configuration III;
the ⅛ model of configuration IV is designed with 1 closed hexagonal hole 5-1, 6 identical closed quadrangular holes 5-2 and 6 identical open triangular holes 5-3, and the holes 5-3 become closed triangular holes 5-4 after symmetry operation in a complete configuration IV; and
the ⅛ model of configuration V is designed with 3 identical closed pentagonal holes 6-1, 3 identical closed hexagonal holes 6-2 and 6 identical open quadrangular holes 6-3, and the holes 6-3 become closed pentagonal holes 6-4 after symmetry operation in a complete configuration V.
